(19) United States
(12) Patent Application Publication
(10) Pub. No.: US 2011/0208075 A1
(43) Pub. Date: Aug. 25, 2011

(54) System and Method for Guide Wire Detection

(75) Inventors: Chad T. Giese, St. Paul, MN (US); H. Toby Markowitz, Roseville, MN (US)

(73) Assignee: Medtronic, Inc., Minneapolis, MN (US)

(21) Appl. No.: 12/712,656

(22) Filed: Feb. 25, 2010

(51) Int. Cl. A61B 5/0452; A61B 5/053

(52) U.S. Cl. 600/509; 600/547

(57) ABSTRACT

A system for detecting a protrusion of a conductor within an anatomy is provided. The system can include a conductor, an insulator and a protrusion module to indicate a protrusion if the conductor protrudes from the insulator within the anatomy. The conductor can comprise a guide wire, a stylet, a pacing lead, a defibrillation lead, or other conductive instruments. The insulator can comprise a catheter, a sheath a cannula or other insulative instruments. The system can detect protrusion based on an impedance or an electrogram measurement.

### FIELD

The present disclosure relates to a system and method for detecting whether a guide wire extends beyond the distal end of a catheter while the distal end of the catheter is within the body of a patient.

### BACKGROUND

A wide variety of cardiac rhythm disorders are treated by electrical sensing and stimulation. Many of these conditions are treated by the use of permanent implantable medical devices (IMD) such as cardiac pacemakers, cardiac defibrillators, and cardiac resynchronization devices. Implanted stimulators are also used in other fields of medicine such as for neurological stimulation of the spinal cord, the brain, and specific nerves such as the vagus nerve. A variety of target organs are treated for the different conditions. Cardiac rhythm management devices provide electrical stimulation to and sensing of the heart.

The modules that contain an apparatus for stimulating and sensing may be referred to as pulse generators. An implantable cardiac rhythm management system such as a pacemaker may consist of a pulse generator and a lead or more than one lead. Leads comprise insulated conductors connecting the pulse generator to an electrode that is in or on a tissue of the heart. The pulse generator is often placed in the pectoral region of a patient and the leads are threaded through veins into the heart of the patient.

The body is conductive as it is comprised largely of water which is conductive due to the presence of electrolytes in solution. As a reminder illustration of the body's conductivity, the electrocardiogram (ECG) is a well known diagnostic test that is possible because the small electrical signals generated by the heart are conducted throughout the body by the various conductive body fluids that comprise the body. The ECG is detected from electrodes on the surface of the body and result from conduction within the body. Various constituent components of the body are conductive but have different conductivities. Blood, for example, is more conductive relative to other body constituents but is less conductive than inorganic metallic conductors such as copper or aluminum. Body fluid is similar in makeup to saline and has conductivity similar to blood. An insulator is a material whose conductivity is poor. Air is one of the least conductive media and as such is a good insulator.

The lead for connecting a pulse generator to a heart electrode may be attached to the outside of the heart. More commonly, such a lead is inserted into a vein in the pectoral region of the patient and navigated through a series of veins into the heart to be located for permanent implant. Most leads have a central lumen which accepts a stylet, a stiff metal rod that may be shaped. A proximal knob on the stylet allows an implanting physician to control and direct the lead to be implanted. The knob may be used to withdraw or advance the stylet. It may also be used to rotate the stylet, thus steering the lead. Some leads have no central lumen and do not utilize a stylet. Leads without a central lumen may be delivered through the veins with the use of a catheter.

The implantation of leads for cardiac pacing and defibrillation through veins to the heart is accomplished with the use of fluoroscopy, a commonly used imaging modality using x-ray radiation. The x-rays used in fluoroscopy are ionizing radiation which has attendant adverse health consequences including the potential for burning skin and raising the risk of certain cancers. Navigation systems using alternative imaging technologies have been developed for cardiac rhythm management purposes including the implantation of cardiac pacing and defibrillation leads. Alternative imaging modalities for lead implantation can eliminate or reduce the use of fluoroscopy as well as reduce the radiation exposure for hospital staff and patients. Two technologies which have found use in cardiac navigation systems are electromagnetic radiation and electrical conduction. Neither of these two technologies risk the injuries encountered with the use of the ionizing radiation employed by fluoroscopy.

Electromagnetic systems used for localization rely upon a transmitter, generally located outside the body and instrumentation placed within the body to receive the electromagnetic radiation. The instruments within the body are connected to a unit outside the body which performs localization and display. Each instrument to be localized should contain one or more electromagnetic sensors. The electromagnetic sensor commonly used is a wire coil. Small diameter devices that are placed within the body such as stylets or guide wires require small coils,

Localization systems that rely on the body's ability to conduct electricity use electrodes attached to the body and instruments placed within the body to receive the electricity. The electrodes within the body are connected to a unit outside the body where localization and display are performed. Each instrument to be localized desirably comprises one or more electrodes. Cardiac rhythm management leads employ electrodes for the sensing and stimulation of cardiac tissue. These electrodes may also be used for localization. Localization systems based on electrical conduction may be referred to as electropotential, impedance or bioimpedance based. The electrodes in the body used for localization sense voltages, sometimes referred to as potentials. The magnitude of the voltage detected by an electrode is based on the electrode's position and the impedance of the patient. Impedance within a body may be referred to as bioimpedance.

Leads used for cardiac rhythm management comprise electrodes but not the coils used by electromagnetic systems. While it might be possible to construct implantable leads with coils, it is unlikely. The addition of coils may require additional conductors to the lead, may add to the cost of manufacture of the lead, and may decrease the reliability of the lead. Implantable leads for cardiac rhythm management face a challenging environment within the body with significant biochemical and biomechanical stresses. The reliability of leads is a major clinical concern which will likely inhibit the incorporation of sensors which are not related to the specific device therapy.

Over-the-wire systems were developed and adopted for interventional procedures such as the placement of balloons and stents. To reach a target vessel with a complicated and tortuous approach, a flexible wire with an atraumatic distal end is placed. A larger, stiffer catheter may be placed over the wire and then fed over-the-wire and to the target. Wires, or guide wires, may gain distal safety through the use of a floppy end or the use of “J” shape at the distal end. The over-the-wire technology was adopted for the implantation of leads in the left ventricular coronary anatomy for cardiac resynchronization therapy. Many permanent implantable pacing leads for the left heart utilize over-the-wire technology, some with stylets.

As stylets are stiff and have a sharp end, it is important they be properly mated to the lead that is being implanted such that they not protrude past the end of an over-the-wire lead. If such a stylet were to protrude past the distal end of an over-the-wire lead, the stylet might penetrate and perforate a vessel well causing vessel wall damage with dire consequences. When placing a wire through an over-the-wire lead, it is important to understand the position of both the lead and the wire. For patient safety, it is important for the physician to be alerted if the wire protrudes beyond the lead end.

While it is possible to make mechanical measurements and mark the wire, such a procedure takes valuable operative time. The resulting marks are difficult to visualize and are difficult to retain in the presence of liquids such as blood, other body fluids or saline used in the operative field, blood, etc. Furthermore, the physician who is implanting such systems must pay attention to various monitors dealing with imaging systems and the physiological monitoring of the patient so can not afford to be distracted watching for markings on the instruments being manipulated in the sterile operative field.

Navigation systems using the electromagnetic technology are challenged to localize permanently implantable pacing leads as the leads do not include electromagnetic sensors. A wire used in an over-the-wire lead system may be instrumented with a coil since the wire is not implanted permanently. With a coil in a guide wire, the navigation system can understand the position of the coil sensor within the wire, even when the wire is totally within the implantable lead. However, a navigation system based solely on electromagnetic localization is unable to know the position of a permanently implantable lead since the lead lacks an electromagnetic sensor. Thus, the electromagnetic based navigation system will be unable to detect if the wire protrudes beyond the end of the lead since a permanently implantable pacing or defibrillation lead can not be instrumented with a coil. As the user must be alert to the relative locations of the wire and the lead, it is important the navigation system inform the user when the lead does not encompass the wire and the wire protrudes past the distal end of the lead.

For navigation systems based on electrical conduction, or bioimpedance, the permanently implantable lead may be localized by virtue of the electrodes on the lead when the electrodes are exposed to blood. However, if the lead is within a protective insulator such as a sheath or when a wire is within a permanently implantable lead, a bioimpedance based navigation system is unable detect the correct location of the lead or the wire since it is not exposed to the blood. Only a very small portion of the blood pool may reach the electrodes on a lead through the end of a sheath. Similarly, a small amount of blood may reach a wire through the end of an over-the-wire lead. A small amount of blood constitutes a high impedance to the flow of electricity and confounds a navigation system based on bioimpedance. Therefore, in such conditions, the bioimpedance based navigation system is unable to localize the wire when the wire does not protrude beyond the end of the lead.

The resistivity of blood is less than that of tissue which is less than that of air. Air is an especially good insulator, it is highly resistive. In the body, in body fluid or in blood, there is good conductivity. An electrode mounted circumferentially on a lead and having good contact with blood will present low impedance when measured with another electrode that is also in good contact with the body or blood.

However, an electrode on a lead wherein the lead is placed within a sheath such as a catheter has only a very small, thin tunnel of blood connecting the electrode with the blood pool. This small thin conduit of blood becomes a high impedance link to the blood pool. Thus, when measured with reference to another electrode in contact with the body or the blood, the impedance to the electrode on the lead is low when the electrode is uncovered and high when the electrode is covered with an insulator such as a sheath or catheter.

### SUMMARY

A system and method are described to detect the protrusion of a conductor from within an insulator.

In one embodiment, a system for detecting protrusion of a conductor in an anatomy comprises an insulator, the insulator introduceable within the anatomy, a conductor, the conductor protrudable from the insulator to the anatomy, the conductor uninsulated distally, and a protrusion module electrically connected to the conductor, the protrusion module indicating a protrusion if the conductor protrudes from the insulator within the anatomy

In another embodiment, a method for detecting protrusion of a conductor in an anatomy comprises providing an insulator, the insulator introduced within an anatomy; providing a conductor, the conductor protrudable from the insulator within the anatomy and uninsulated distally, performing an electrical measurement with the conductor, and indicating a protrusion of the conductor from the insulator based on the electrical measurement if the conductor protrudes from the insulator within the anatomy of the patient.

In certain embodiments, the conductor may comprise a guide wire, a stylet, a pacing lead, a defibrillation lead, a neurological stimulation lead, a temporary pacing wire or a permanently implantable pacing lead.

In certain embodiments, the insulator may comprise a catheter, a cannula, a pacing lead, a defibrillation lead, a balloon catheter or an over-the-wire instrument.

In other embodiments, the protrusion module indicates protrusion based on an electrical measurement of an electrogram signal or an impedance measurement.

In another embodiment, the system comprises a protrusion module connected to a navigation system to display an icon indicating protrusion or no protrusion of the conductor.

### BRIEF DESCRIPTION OF THE DRAWINGS

FIG. 1a depicts an insulator partly within an anatomy and a conductor within the insulator;

FIG. 1b depicts an insulator partly within in an anatomy and a conductor protruding from the insulator to the anatomy;

FIG. 2a depicts an insulator wholly in an anatomy and a conductor within the insulator;

FIG. 2b depicts an insulator wholly within an anatomy and a portion of the conductor protruding from the insulator;

FIG. 3 depicts an insulative member and a conductive member within the insulative member;

FIG. 4 depicts an insulative member and a portion of a conductive member protruding from the insulative member;

FIG. 5 depicts an insulative member and a lead having one lead electrode within the insulative member;

FIG. 6 depicts an insulative member and a lead having one lead electrode protruding from the insulative member;

FIG. 7 depicts an insulative member and a lead having two lead electrodes protruding from the insulative member;

FIG. 8 depicts an insulative member and a lead having two lead electrodes wherein a protrusion of a conductor would be indicated for the distal lead electrode and protrusion would not be indicated for the proximal lead electrode;

FIG. 9 illustrates an exemplary embodiment of the invention in use;

FIG. 10 is an exemplary representation of a schematic diagram of a protrusion module based on a measured impedance;

FIG. 11 is an exemplary flow diagram for determining protrusion of a conductor based on a measured impedance;

FIG. 12 is an exemplary block diagram of a protrusion module based on an electrogram measurement; and

FIG. 13 is an exemplary schematic diagram of a protrusion module connected to a cardiac navigation system and a display of icons.

### DETAILED DESCRIPTION

The following description is merely exemplary in nature and is not intended to limit the present disclosure, application, or uses. It should be understood that throughout the drawings, corresponding reference numerals indicate like or corresponding parts and features. As indicated above, the present teachings are directed towards providing a system and method for detecting a guide wire. It should be noted, however, that the present teachings could be applicable to any appropriate procedure in which it is desirable to determine a position of a conductive member within an insulator. Therefore, it will be understood that the following discussions are not intended to limit the scope of the appended claims.

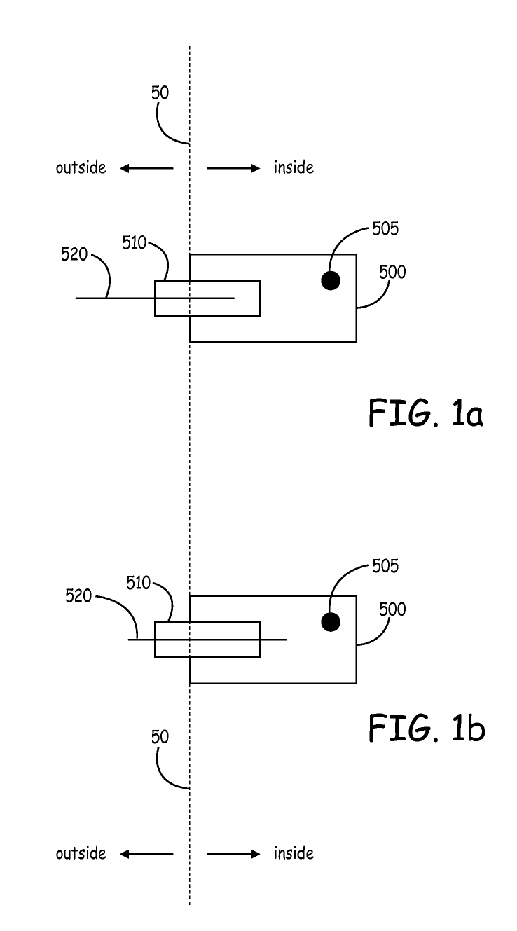

FIGS. 1a-b, and 2a-b illustrate an anatomy 500, an insulator 510 and a conductor 520. The anatomy 500 models a conductive member filled with fluid or blood into which various elements can be introduced. An anatomical electrode 505 is on or in the anatomy 500 and has contact with the fluid, the blood or a tissue of the anatomy 500.

The insulator 510 models a catheter, or sheath. In FIGS. 1a and 1b, a catheter is introduced into the anatomy 500 with a portion remaining outside the anatomy. The demarcation of the percutaneous access 50 models the portion of the insulator 510 which is outside the anatomy, to the left in the figures and that portion which is inside the anatomy, to the right in the figures, as labeled. The conductor 520 models a guide wire, a stylet or other conductive instrument which may be introduced within the anatomy 500. In FIG. 1a, the conductor is introduced within the insulator but the conductor does not protrude beyond the insulator into the anatomy. The impedance measured between the conductor 520 and the anatomical electrode 505 will be high (not less than 500 ohms) because the conductor is not exposed to the blood, the fluid or the tissue of the anatomy 500.

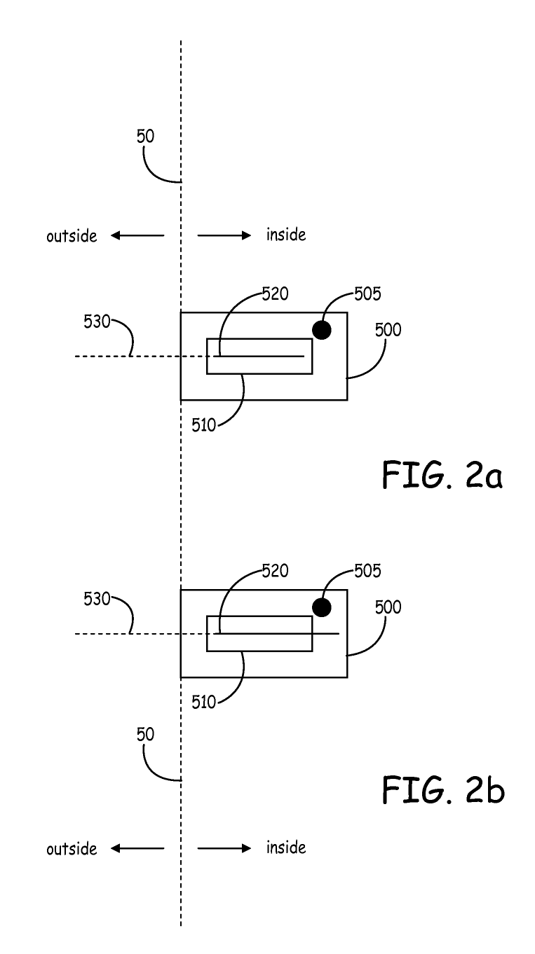

In FIG. 1b, the conductor 520 is introduced within the insulator, however, unlike the embodiment depicted in FIG. 1a, the conductor does protrude beyond the insulator within the anatomy. The impedance measured between the conductor 520 and the anatomical electrode 505 will be low (less than 500 ohms) because the conductor has exposure to the blood, fluid or tissue of the anatomy 500. FIGS. 2a and 2b show the insulator 510 totally within the anatomy. For these embodiments, the insulator 510 models a totally implantable device such as a permanently implantable pacemaker. The conductor 520 could be any conductor that is protrudable, i.e. is able to protrude, from the insulator 510, e.g. the implantable pacemaker. FIGS. 2a and 2b model a totally implantable device but with an insulated conductor 530 connected to the indwelling conductor 520 such as might occur during delivery of an implantable medical device such as a leadless pacemaker. The insulated conductor can connect to instrumentation that is external to the anatomy 500 without exposure to the blood, body fluid or tissue of the anatomy. The impedance between the insulated conductor 530 and the anatomical electrode 505 in the embodiment modeled in FIG. 2a would be high because the conductor 520 is not exposed to the anatomy. The impedance between the insulated conductor 530 and the anatomical electrode 505 in the embodiment of FIG. 2b would be low because the conductor is exposed to the anatomy; the conductor protrudes from the insulator 510.

Table 1 summarizes the key characteristics of the models shown in FIGS. 1a-b and 2a-b. The impedance measured between the conductor 520 and the anatomical electrode 505 which is in or on the anatomy 500, is low for each situation in which the conductor 520 protrudes from the insulator 510 within the anatomy 500. The impedance is high for each situation in which the conductor does not protrude from the insulator within the anatomy.

TABLE 1

| | Conductor | Exposed to | |
|---|---|---|---|
| FIG. | Protruding? | anatomy? | Impedance |
| 1a | NO | NO | HIGH |
| 1b | YES | YES | LOW |
| 2a | NO | NO | HIGH |
| 2b | YES | YES | LOW |

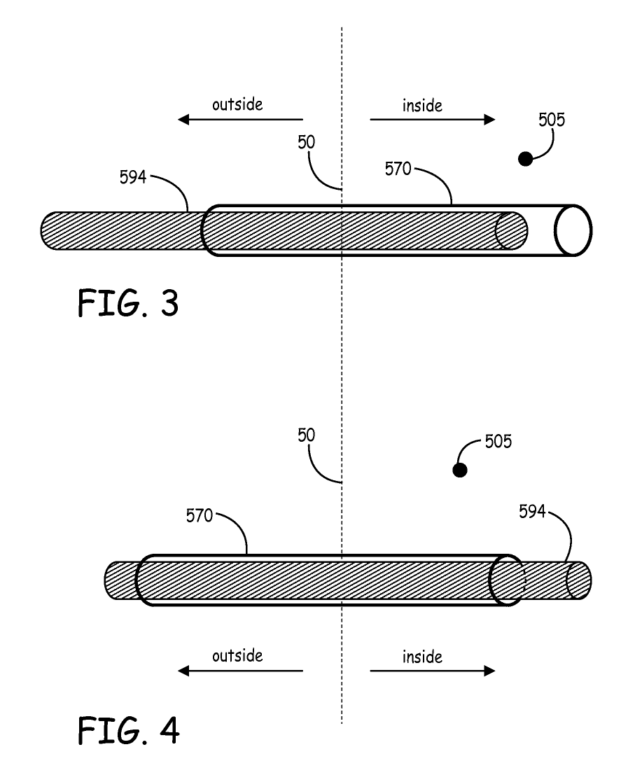

FIGS. 3 and 4 illustrate an insulative member 570, wherein the insulative member models a sheath indwelling in the anatomy but a portion of the sheath outside the anatomy. The insulative member contains a conductive member 594. The insulative member models a guide wire which is conductive. While guide wires may be constructed with an insulative coating, the insulative member 594 should be uncoated or uninsulated in the distal region that is protrudable from the sheath and may be exposed to the anatomy. The conductive member 594 is uninsulated distally. In FIG. 3, the conductive member is within the insulative member. The blood, body fluid and tissue of the anatomy are exposed only to the cross-sectional area at the end of the conductive member. In contrast, a larger surface area of the conductive member is exposed in FIG. 4. The insulative member need only be large enough to allow the conductive member to slide within the insulative member. A small radial distance between the conductive member and the insulative member does not prevent blood from entering the region, however, the small thin layer of blood between the insulative member and the conductive member presents a high impedance to the blood pool outside the insulative member. The measurement of impedance from conductive member 594 to the anatomical electrode 505 in or on the anatomy will reflect the impedances modeled in FIGS. 1a-b and 2a-b.

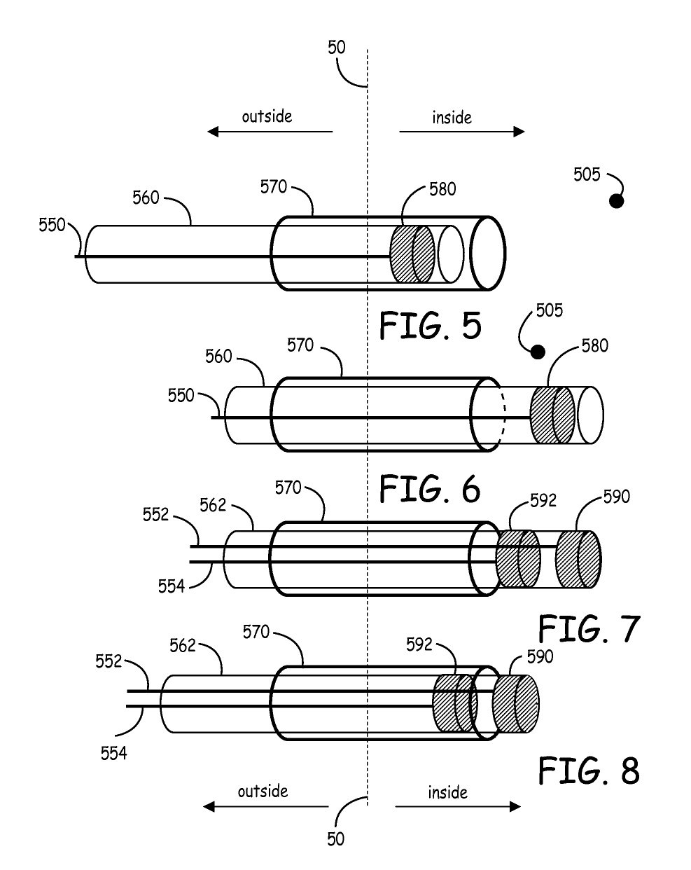

A unipolar pacing lead is illustrated in FIGS. 5 and 6 and a bipolar pacing lead is illustrated in FIGS. 7 and 8. A unipolar pacing lead comprises a single circumferential lead electrode 580 and relies upon a second, separate, electrode for pacing and sensing. Impedance measured between the anatomical electrode 505 which is in or on the anatomy and circumferential lead electrode 580 will be high in the embodiment depicted in FIG. 5 because the lead electrode is within the insulative member 570 and low in FIG. 6 because the lead electrode protrudes from the insulative member 570.

The bipolar pacing lead shown in FIGS. 7 and 8 comprises a distal circumferential lead electrode 590 and a proximal circumferential lead electrode 592. Impedance may be measured between the distal and the proximal lead electrodes to determine the concurrent protrusion of both the distal and proximal lead electrodes. Impedance may also be measured between the distal lead electrode and the anatomical electrode 505 to determine protrusion of the distal lead electrode. Furthermore, impedance may be measured between the proximal lead electrode and the anatomical electrode 505 to determine protrusion of the proximal lead electrode. When the impedance is measured between the distal and proximal lead electrodes, the embodiment shown in FIG. 7 will result in low impedance because both lead electrodes 590, 592 protrude from the insulative member 570. The embodiment shown in FIG. 8 would result in high impedance because only the distal circumferential lead electrode 590 protrudes from the insulative member and the proximal circumferential lead electrode 592 is within the insulative member.

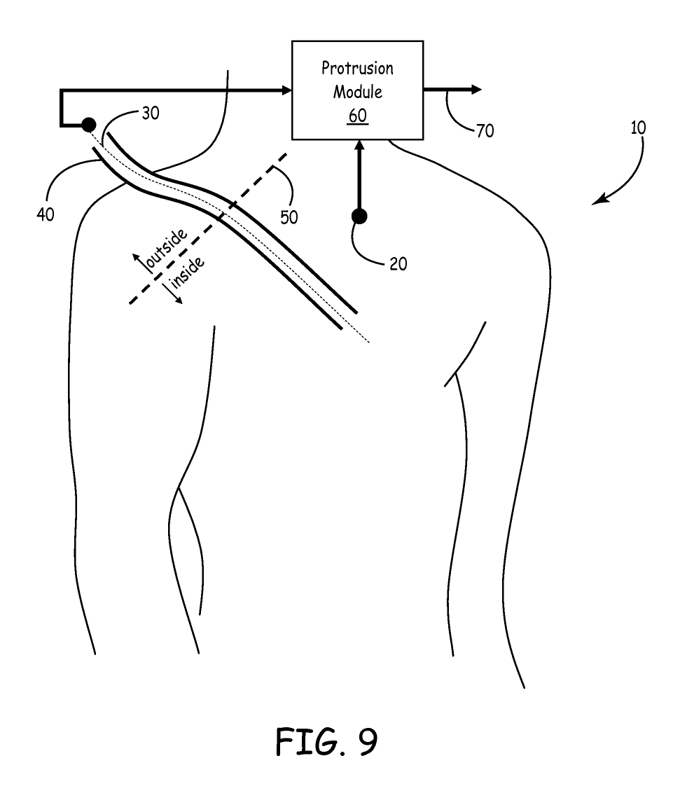

FIG. 9 illustrates a patient 10 with an indwelling catheter 40. The catheter enters the anatomy or body at the demarcation of the percutaneous access 50. A guide wire 30 is within the catheter and is electrically connected to a protrusion module 60. A patient electrode 20 that is on or in the patient 10, is also electrically connected to the protrusion module 60. The protrusion module has as output, an indicator signal 70. The indicator signal may be electrical or may be converted to other means of indication such as a mechanical indication, an optical signal such as from a source of light, an audible indication or an icon on an information display. This illustrates a system for detecting protrusion of a conductor in an anatomy comprising an insulator, the insulator introduced within the anatomy; a conductor, the conductor protrudable from the insulator within the anatomy and the conductor uninsulated distally; and a protrusion module electrically connected to the conductor, the protrusion module indicating a protrusion if the conductor protrudes from the insulator within the anatomy.

The conductor described in these embodiments could be a guide wire, a stylet, a pacing lead, a defibrillation lead, a neurological stimulation lead, a temporary pacing wire or a permanently implantable pacing lead. The insulator used in these embodiments could be a catheter, a cannula, a sheath, a tube, an introducer, an over-the-wire pacing lead, an over-the-wire defibrillation lead, an over-the-wire neurological stimulation lead an over-the-wire balloon catheter or any over the wire instrument. A lead used for stimulation and/or sensing comprises one or more conductors, a proximal connection (or connections) and lead electrodes disposed on a lead body portion that will reside in the anatomy. A lead comprises an insulated conductor. The lead electrodes are electrically connected to the conductor and may contact the blood, the body fluid or the tissue of an anatomy. The lead may contain a lumen which is closed to the anatomy. A lead described in this paragraph does not refer to the over-the-wire construction which is described below. The lead described in this paragraph has the properties of a conductor described herein.

An over-the wire lead used for stimulation and/or sensing differs from a lead of the above paragraph in that it contains a lumen which is open to the anatomy at the distal end of the lead. A conductor placed within the over-the-wire lumen may be protrudable from the lead. If the lead is placed in the anatomy, a conductor placed within the lumen may protrude to the anatomy. An over-the-wire lead may act as an insulator to a conductor placed within the lumen of the over-the-wire lead. An over-the-wire lead may contain conductors, lead electrodes and insulation. Thus, an over-the-wire lead may exhibit the properties of an insulator with respect to a conductor placed within the lumen of the over-the-wire lead; an over-the-wire lead may exhibit the properties of a conductor with lead electrodes disposed on the over-the-wire lead. An over-the-wire lead behaving as a conductor may be contained within an insulator such as a sheath, an introducer, a catheter, or the like.

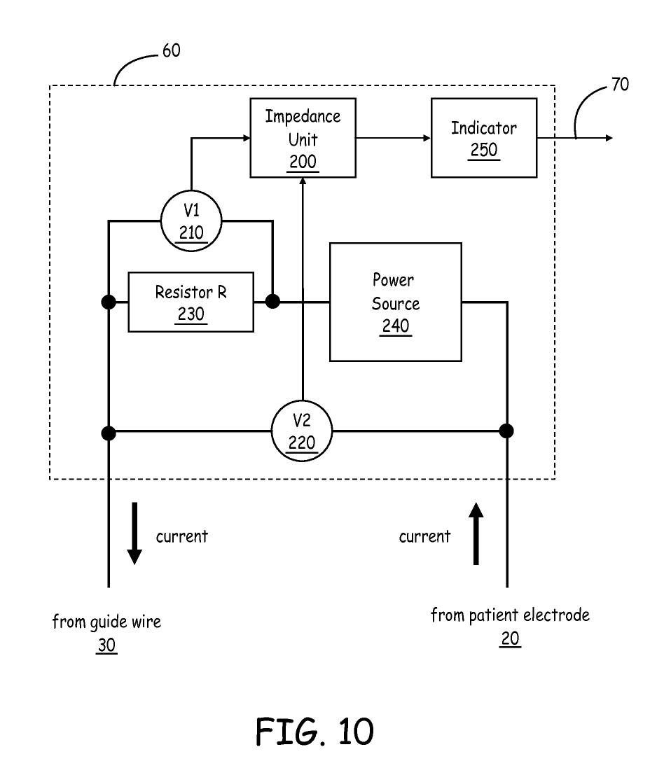

FIG. 10 illustrates a schematic of an embodiment to detect protrusion of a conductor based on a measured impedance. The protrusion module 60 is connected to the guide wire 30 and the patient electrode 20. The protrusion module measures the impedance between the guide wire and the patient electrode. The impedance may be measured continuously or the impedance may be measured at select times, i.e. it may be sampled. The power source 240 provides a current to flow through the patient electrode and the guide wire. The voltage measurement V2 220 indicates the voltage applied between the guide wire 30 and the patient electrode 20. The current through the guide wire 30 and the patient electrode 20 is measured by measuring the voltage across a resistor R 230 used to sample the current. The measured voltage across the resistor R allows computing the current flowing by ohm's law. The resistor R 230 is chosen to be small compared to the expected resistance or impedance between the guide wire and the patient electrode. The current flowing is the measured voltage, V1 in volts divided by the resistance R in ohms.

The measurement V2 in FIG. 10 is the applied voltage. The impedance unit 200 divides the measured voltage, V2, by the computed current, to derive the impedance presented to the system by the guide wire 30 and the patient electrode 20. The indicator 250 derives its input from the impedance unit 200 and outputs the indicator signal 70.

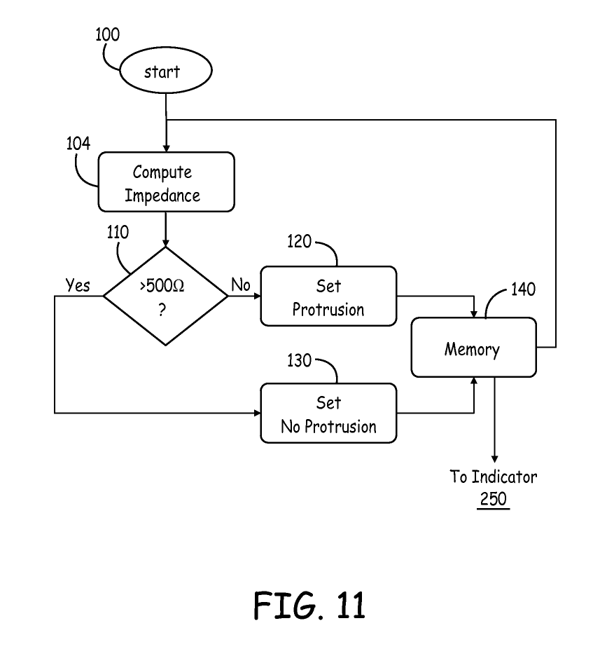

FIG. 11 illustrates a flow diagram of a process within the impedance unit 200 (FIG. 10) to determine protrusion based on a measured impedance. The process begins in step 100. In step 104, the impedance is computed as:

Current=V1/R

and

Impedance=V2/Current=V2/(V1/R)

In step 110, the system checks whether the measured impedance is greater than 500 ohms. If yes, the impedance is greater than 500 ohms, the system declares this to be no protrusion in step 130. The memory is set to no protrusion in step 140 and the process returns to measuring the impedance in step 110. If the impedance does not measure greater than 500 ohms in step 110, protrusion is declared in step 120 and the memory is set to protrusion. The contents of the memory provide a protrusion status to the indicator 250 (FIG. 10, described above).

In another embodiment, protrusion of the conductor from an insulator may be determined based on a measured electrogram and detection of P-waves, R-waves or T-waves if the distal end of the insulator resides in the heart of a patient. In the electrocardiogram, the P-wave represents depolarization of the atria, the R-wave the ventricles and the T-wave, repolarization of the ventricles. An electrogram is a signal from electrodes inside the heart. While the electrogram does not contain P, R and T waves, it does contain elements reflecting action of the atria and the ventricles depending on the location of the electrode from which the electrogram is recorded. In this application, P-waves shall mean a signal reflecting depolarization of the atria; R-waves shall mean a signal reflecting depolarization of the ventricles; T-waves shall mean a signal reflecting repolarization of the ventricles.

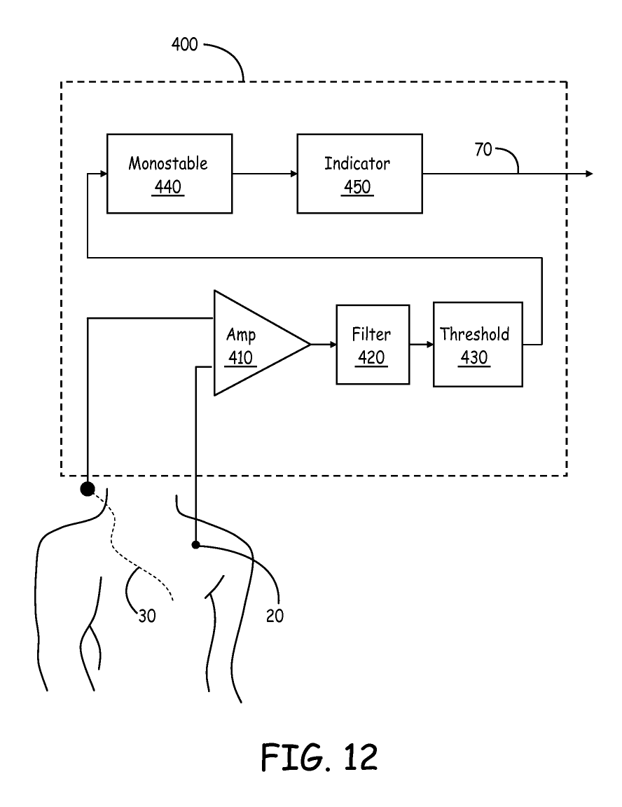

FIG. 12 illustrates a block diagram of the electrogram based protrusion module 400. The electrogram based protrusion module 400 may be substituted for the protrusion module 60 in the embodiment illustrated in FIG. 9 or in the embodiment illustrated in FIG. 14 (described below). The protrusion module 400 is connected to the guide wire 30 and the patient electrode 20. These two electrically connect to the electrogram amplifier 410 (“Amp”). The electrogram filter 420 (“Filter”) allows P-wave, R-wave and T-wave signals to pass. The threshold detector 430 (“Threshold”) allows detection in the presence of P-waves, R-waves or T-waves but does not allow detection if the P, R or T-waves are not present. If a P-wave, an R-wave or a T-wave is detected, the monostable 440 is triggered for 1.2 seconds. A timer is necessary since the occurrence of electrocardiographic or electrogram features is periodic, not constant. The timer is set to correspond to the slowest rate expected during the setting of lead implantation, about 50 beats per minute or 1.2 seconds. The monostable output is sent to the indicator 450 which outputs the indicator signal 70. The monostable 440 may be retriggered by subsequent sensing of a P-wave, an R-wave or a T-wave.

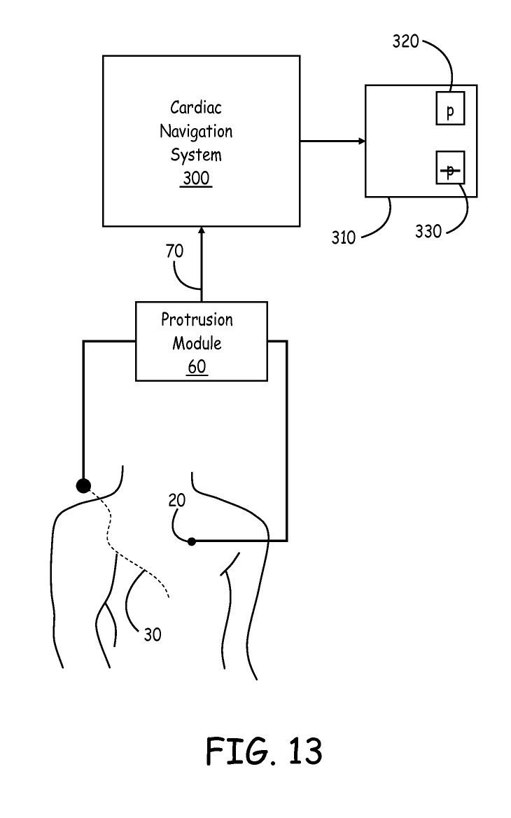

A protrusion module may be used in conjunction with an imaging system or a cardiac navigation system such as illustrated in FIG. 13. The protrusion module 60 is connected to, the guide wire 30 and the patient electrode 20. From the protrusion module 60, the indicator signal 70 is connected to the cardiac navigation system 300. The navigation system provides a cardiac navigation display 310 for the physician performing a lead implantation or other like procedure in which information about the protrusion of a guide wire 30 is important. The display can show the cardiac navigation protrusion icon 320 when the guide wire is protruding. The icon 320 may be removed when the icon does not protrude. Additionally, the display can show cardiac navigation no protrusion icon 330 when the guide wire does not protrude and remove icon 330 when the guide wire does protrude. In this manner, the physician has easy visual access to important imaging information and the protrusion indication on one display.

The embodiments described herein illustrate detection of protrusion based on electrical measurements, measurements of impedance and electrogram measurements.

While the position of the guide wire 30 has been described as being determined based on a sensed impedance, those of ordinary skill in the art will appreciate that the present disclosure, in its broadest aspects, may be constructed to include measurement of voltage or current.

What is claimed is:

1. A system for detecting protrusion of a conductor in an anatomy comprising:
an insulator, the insulator introduceable within the anatomy;
a conductor, the conductor protrudable from the insulator to the anatomy, the conductor uninsulated distally; and
a protrusion module electrically connected to the conductor, the protrusion module indicating a protrusion if the conductor protrudes from the insulator within the anatomy.

2. The system of claim 1 wherein the conductor comprises:
one of a guide wire, a stylet, a pacing lead, a defibrillation lead, a neurological stimulation lead, a temporary pacing wire, or a permanently implantable pacing lead.

3. The system of claim 1 wherein the insulator comprises:
one of a catheter, a cannula, a sheath, a tube, an introducer, an over-the-wire pacing lead, an over-the-wire defibrillation lead, an over-the-wire balloon catheter, an over-the-wire neurological stimulation lead or an over-the-wire instrument.

4. The system of claim 1 wherein the protrusion module indicates the protrusion based on an electrical measurement.

5. The system of claim 4 wherein the electrical measurement comprises an electrogram signal detection or an impedance measurement.

6. The system of claim 5 wherein the electrogram signal detection comprises a detection of P-waves, a detection of R-waves, or a detection of T-waves.

7. The system of claim 5, wherein the impedance measurement is measured continuously, or the impedance measurement is measured at select times.

8. The system of claim 4, wherein an indication of protrusion occurs if an impedance measurement is less than 500 ohms and an indication of no protrusion occurs if the impedance measurement is not less than 500 ohms.

9. The system of claim 1 further comprising a navigation system operably connected to the protrusion module.

10. The system of claim 9, wherein the navigation system comprises a display of an icon to represent the protrusion of the conductor if the protrusion module is indicating the protrusion of the conductor, or, a display of an icon to represent no protrusion of the conductor if the protrusion module is not indicating the protrusion of the conductor.

11. A method for detecting protrusion of a conductor in an anatomy comprising:
providing an insulator, the insulator introduced within an anatomy;
providing a conductor, the conductor protrudable from the insulator within the anatomy and uninsulated distally;
performing an electrical measurement with the conductor; and
indicating a protrusion of the conductor from the insulator based on the electrical measurement if the conductor protrudes from the insulator within the anatomy of the patient.

12. The method of claim 11, wherein providing the insulator further comprises:
providing one of a catheter, a cannula, a sheath, a tube, an over-the-wire pacing lead, an over-the-wire defibrillation lead, over-the-wire neurological stimulation lead, an over-the-wire balloon catheter and an introducer.

13. The method of claim 11, wherein the conductor is one of a guide wire, a stylet, a pacing lead, a defibrillation lead, a neurological stimulation lead, a temporary pacing wire, or a permanently implantable pacing lead.

14. The method of claim 11 wherein performing the electrical measurement comprises:
detecting an electrogram signal, or measuring an impedance.

15. The method of claim 14 wherein detecting the electrogram signal comprises:
detecting P-waves, detecting R-waves or detecting T-waves.

16. The method of claim 14 wherein measuring the impedance comprises measuring the impedance continuously or measuring the impedance at select time intervals.

17. A medical system comprising:
means for insulating within an anatomy;
means for conducting within an anatomy;
means for the conducting means to protrude from the insulating means;
means for electrical measuring;
means for electrically connecting the electrical measuring means to the conducting means;
means for detecting the conducting means protruding from the insulating means; and
means for indicating a protrusion of the conducting means from the insulating means if the insulating means protrudes from the conducting means within the anatomy.

18. The system of claim 17 wherein the means for electrical measuring comprises means for measuring an impedance or means for measuring an electrogram.

19. The system of claim 18 wherein the means for measuring the impedance comprises
means to determine if the impedance is greater than 500 ohms;
means for indicating protrusion
if the impedance is greater than 500 ohms, and,
means for indicating no protrusion
if the impedance is not greater than 500 ohms.

20. The system of claim 18 wherein the means for measuring an electrogram comprises:
means to detect P-waves,
means to detect R-waves, or
means to detect T-waves.

21. The system of claim 17 further comprising:
means for navigation within the anatomy,
means for connecting the navigation means to the indicating means,
means for displaying a protrusion icon
if the indicating means indicates the protrusion, and
means for displaying a no protrusion icon
if the indicating means indicates no protrusion.
